(19) United States
(12) Patent Application Publication
(10) Pub. No.: US 2007/0015218 A1
(43) Pub. Date: Jan. 18, 2007

(54) A Method for the Separation Anti-Amyloid Beta Antibody with Amyloid Beta Peptide

(75) Inventors: Chuanhai Cao, Tampa, FL (US); Qingyou Li, Tampa, FL (US); David G. Morgan, Clearwater, FL (US)

Correspondence Address: SMITH HOPEN, PA, 180 PINE AVENUE NORTH, OLDSMAR, FL, 34677, US

(73) Assignee: University of South Florida, Tampa, FL (US)

(21) Appl. No.: 11/279,828

(22) Filed: Apr. 14, 2006

Related U.S. Application Data
(63) Continuation of application No. PCT/US04/33748, filed on Oct. 14, 2004.
(60) Provisional application No. 60/481,505, filed on Oct. 14, 2003.

(51) Int. Cl. G01N 33/567; G01N 33/53

(52) U.S. Cl. 435/7.2

(57) ABSTRACT

The present invention is a method of effectively dissociating an antibody and antigen from the complex they form. The method enables researchers to accurately identify anti-amyloid beta peptide and its antibody from sera samples. It can also be used for the evaluation of the outcome of Alzheimer's patient treatment based on the amyloid peptide load and antibody level in the sera.

### CROSS-REFERENCE TO RELATED DISCLOSURE

This application claims the benefit of international patent application number PCT/US2004/033748, filed Oct. 14, 2004, which claims the benefit of U.S. Provisional Patent Application Ser. No. 60/481,505, filed Oct. 14, 2003, which are fully incorporated herein by reference.

### GOVERNMENT RIGHTS

Research relating to the present application was supported by NIH grants AG 18478 and AG 20227. Accordingly, the U.S. federal government may have rights in the present invention.

### BACKGROUND OF THE INVENTION

Immunization against amyloid-beta has been suggested as a possible preventive or therapeutic treatment for Alzheimer's disease. Vaccines directed against the Aβ peptide reduce amyloid loads in amyloid precursor protein (APP) transgenic mice and protect mice from amyloid-associated memory impairments. Although a fraction of patients in a clinical Aβ vaccination trial developed adverse reactions, there are indications that some patients benefited from the immunization. Thus, although reformulation may be necessary, some form of anti-Aβ immunotherapy may still be a useful treatment for Alzheimer's Disease (AD).

It has been noted that reduced antibody titers in mice transgenic for human APP compared to nontransgenic mice. Typically, this was attributed to some form of self-tolerance that could be partially overcome with additional immunizations. One approach to overcoming B cell tolerance to self proteins when producing vaccines has been to conjugate the self-antigen at high density to papillomavirus virus-like particles (VLPs).

Therefore, what is needed is a method of overcoming antigen masking of the presence of an antibody in a sample.

### SUMMARY OF INVENTION

In one embodiment, the inventive method includes a procedure for the dissociation of an antibody (here, the anti-Aβ antibody) from an endogenous antigen (Aβ in) serum where a sample is diluted 1:100 with a dissociation buffer (such as PBS buffer with 1.5% BSA and 0.2 M glycine-acetate pH 2.5), to a 500 μl final volume and incubated for 20 minutes at room temperature. The sera is then pipetted into the sample reservoir of Microcon centrifugal filter device, YM-10 (10,000 MW cut-off, Millipore) and centrifuged at 8,000×g for 20 min. at room temperature. The sample reservoir is then separated from the flow through, placed inverted into a second tube and centrifuged at 1000×g for 3 min.

In another embodiment of the present invention, an improved assay is provided for determining the presence of an antibody (anti-Aβ antibodies) in a sample. In this embodiment, a solution wherein the target (anti-Aβ) antibody is dissociated from the antigen (Aβ) peptide and adjusted to pH 7.0 with 15 μl of 1 M Tris buffer, pH 9.0. The retentate volume is brought to the initial volume (500 μl) with ELISA dilution buffer (PBS with 1.5% BSA and 0.1% Tween-20, pH 7.0). The collected sera are then added to an ELISA plate at multiple dilutions to determine the limiting antibody titer. For non-dissociated sera values, the same serum is treated with an identical process except using dissociation buffer, pH 7.0 instead of dissociation buffer, pH 2.5.

### BRIEF DESCRIPTION OF THE DRAWINGS

For a fuller understanding of the nature and objects of the invention, reference should be made to the following detailed description, taken in connection with the accompanying drawings, in which:

FIG. 1. Transgenic APP mice immunized with Aβ vaccines have increased titers when incubated at pH 2.5. Results are from sera obtained 14 days after the fourth vaccination. Aliquots from each sample were incubated at either pH 2.5 or pH 7.0 for 20 minutes, separated by centrifugation through 10,000 MW filters and neutralized for standard ELISA assay. Data presented are mean±sem. * Indicates P<0.001 compared to pH 7.0 (values at pH 7 are 200 and 400 for WT and VLP respectively). Sample size is 6 per group.

FIG. 2. Non-transgenic mice immunized with AB vaccines do not have increased titers when incubated at pH 2.5. Results are from sera obtained 14 days after the fourth vaccination. Aliquots from each sample were incubated at either pH 2.5 or pH 7.0 for 20 minutes, separated by centrifugation through 10,000 MW filters and neutralized for standard ELISA assay. Data presented are mean±sem. Sample size is 6 per group.

FIG. 3. Comparison of anti-Aβ titers of Aβ vaccinated APP and non-transgenic mice after acid dissociation of Aβ and anti-Aβ antibody. Results are from sera obtained 14 days after the fourth vaccination. All samples were incubated at pH 2.5, centrifuged through 10,000 MW filters and neutralized before ELISA assay. Data presented are mean±sem. * P<0.05 compared to APP mice given the same vaccine. Sample size is 6 per group.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

The concept of self-tolerance in Tg2576 derived APP transgenic mice was tested by vaccinating them with the wild-type human Aβ, a wild-type human Aβ conjugated to papillomavirus virus-like particles, and the human AB sequence containing the “Dutch Mutation,” E22Q. In evaluating the initial results the inventors found very low anti-AB titers in the transgenic mice compared to the non-transgenic mice for the wild-type and virus-like particle vaccine preparations, but not the Aβ E22Q preparations, which resulted in very high titers in all mice. Dissociating antibody-antigen complexes with low pH increased the anti-Aβ antibody titers 20-40 fold in APP mice but not in transgenic mice. After dissociation, the anti-Aβ titers were still lower in transgenic mice vaccinated with wild-type of Dutch mutation Aβ vaccines. However, the virus-like particle vaccine appeared to break self-tolerance as no genotype differences remained. The inventors conclude that circulating human Aβ can interfere with ELISA assay measurements of anti-Aβ titers, that virus-like particle vaccines for Aβ avoid self-tolerance and that vaccines with the Dutch mutation act as superantigens provoking a much higher antibody titer than the wild-type antigen.

Vaccines against the Aβ peptide reduce amyloid loads in amyloid precursor protein (APP) transgenic mice and protect mice from amyloid-associated memory impairments. Although a fraction of patients in a clinical trial developed adverse reactions, there are indications that some patients benefited from the immunization. Thus, although reformulation may be necessary, some form of anti-Aβ immunotherapy may still be a useful treatment for Alzheimer's Disease (AD).

Several groups, including that of the present inventors, have noted a reduced antibody titer in mice transgenic for human APP compared to non-transgenic mice. Typically, this was attributed to some form of self-tolerance that could be overcome with additional immunizations of more potent vaccine preparations. Another approach to overcoming tolerance to self proteins when producing vaccines has been to conjugate the antigen to a papillomavirus virus-like particle (VLP). These were compared to a human Aβ variant believed responsible for hereditary cerebral hemorrhage with amyloidosis Dutch-type, which might contain epitopes not present in the wild-type human Aβ vaccine.

Vaccination Protocols

The Tg2576 APP transgenic mice and non-transgenic littermates (produced as described in (Holcomb et al., 1998) were vaccinated with human Aβ1-42 E22Q (Dutch mutant peptide; DM) from American peptide, wild type (WT) Aβ1-42 peptide (American peptide) or a pappilomavirus viral-like particle conjugated to wild type human Aβ 1-40 peptide (VLP,). Vaccines were prepared. For WT and DM, Aβ peptides were suspended in pyrogen-free Type I water at 2.2 mg/ml then mixed with 10× PBS to yield 1×PBS solution and incubated overnight at 37° C. The following day, two volumes of 1×PBS was added to dilute the Aβ peptides further, and then the peptide suspension was emulsified with an equal volume of Freund's complete adjuvant (Sigma). The vaccine preparation (100 μg Aβ/300 μl volume) was injected into each mouse subcutaneously. For the VLP material, 130 μl complete Freund's adjuvant was added to 170 μl VLP preparation conatining 5.6 μg Aβ, then emulsified and injected as 300 μl into each mouse. For the second immunization, the same materials were prepared freshly in incomplete Freund's adjuvant (Sigma) at 14 days after first injection. The third and fourth boosts were made using incomplete Freund's at monthly intervals after the second immunization. Six transgenic and six non-transgenic mice for each group were vaccinated beginning at 9 months of age and sacrificed at 12 months of age, 14 days after the fourth inoculation. Sera were collected under anesthesia by retro-orbital puncture two weeks after the second and third inoculations and by ocular enucleation at sacrifice.

Dissociation of Anti-Aβ Antibody from Endogenous Aβ

Sera were diluted 1:100 with dissociation buffer (PBS buffer with 1.5% BSA and 0.2 M glycine-acetate pH 2.5), to a 500 μl final volume and incubated for 20 minutes at room temperature. The sera were then pipetted into the sample reservoir of Microcon centrifugal filter device, YM-10 (10,000 MW cut-off, Millipore) and centrifuged at 8,000×g for 20 minutes at room temperature. The sample reservoir was then separated from the flow through, placed inverted into a second tube and centrifuged at 1000×g for 3 minutes. The collected solution containing the antibody dissociated from the Aβ peptide was brought to an adjusted pH 7.0 with 15 μl of 1 M Tris buffer, pH 9.0. The retentate volume was bought to the initial volume (500 μl) with ELISA dilution buffer (PBS with 1.5% BSA and 0.1% Tween-20, pH 7.0). The collected sera were then added to an ELISA plate at multiple dilutions to determine the limiting antibody titer. For non-dissociated sera values, the same serum was treated with an identical process except using dissociation buffer, pH 7.0 instead of dissociation buffer, pH 2.5.

An alternate embodiment of the present invention is the use of the dissociation buffer to other antibody/antigen complexes which cannot be readily associated using other methods. Such applications are can be clearly practiced using the present invention are within the scope of the present invention. Variations on the buffers are ranges of lower pH which are considered “acidic” or any pH which is less than about a pH of 7.0. Other acids can readily be substituted for the glycine-acetate such as hydrochloric acid, acetic acid, etc. Possible reagents which can be used and are not meant to be limiting in this invention can include guanidium thiocyanate, betamercaptoethanol, or dithiothreitol for example. Therefore, it would be readily apparent to one of ordinary skill in the art to modify the buffer as deemed necessary in application of the antigen/antibody complexes which requires dissociation for analysis and detection.

Measurements of Antibody Titers

The above dissociated sera were assayed by ELISA for antibody titer. Ninety-six-well Immulon 4HBX plates (Dynex) were coated with 50 μl per well of WT Aβ 1-42 peptide at 5 μg/ml in PBS buffer, pH 7.0 and incubated overnight at 4° C. The plates were washed five times with 0.45% BSA +0.05% Tween-20 (washing buffer, WB) and blocked at 37° C. for 1 hour with blocking buffer (1.5% BSA and 0.05% Tween-20 in PBS). After five washes, the sera were added in duplicate at an initial dilution of 1:100 and diluted two fold serially in blocking buffer and incubated for 1 hour at 37° C. The plates were washed 10 times and anti-mouse IgG conjugated with horseradish peroxidase (HRP) (Sigma Chemical Co. St. Louis, Mo.) diluted 1:5000 was added to the plates and incubated for 1 hour at 37° C. The plates were then washed ten times and developed with 3′,3′,5′,5′-Tetramethylbenzidine (TMB; Sigma). The reaction was stopped with 2M sulfuric acid. The plates were analyzed spectrophotometrically at 450 nm.

Initial ELISA assays were performed without dissociation using standard procedures and resulted in almost undetectable titers in the transgenic mice inoculated with WT or VLP Aβ. However, non-transgenic mice did exhibit readily measurable titers. For the DM Aβ vaccine very high titers were detected in both genotypes. This prompted an inquiry as to whether circulating human Aβ might be masking the antibodies in the transgenic mice. Several methods of dissociating antibodies from their antigens were compared including dithiothreitol (100 mM), β-mercaptoethanol (0.5%) and reduced pH (pH 2.5 as described in methods). The acid dissociation procedure resulted in the greatest increase in anti-Aβ antibody titers (4 fold greater than any of the other treatments). Final sera were collected and compared from all mice when treated at pH 2.5 and pH 7.0 as described in methods.

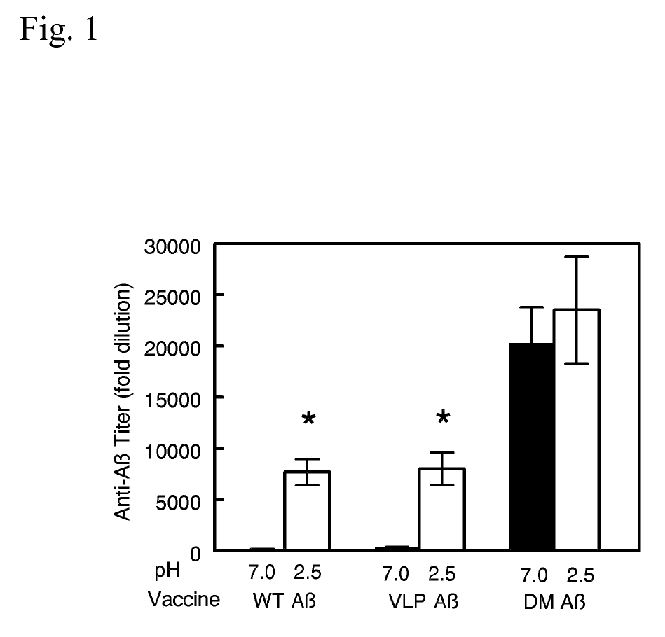

The low pH dissociation procedure caused a dramatic elevation of the apparent anti-Aβ antibody titers in sera collected from transgenic mice (FIG. 1). The titers increased from values near zero in the pH 7.0 condition to roughly 8000 in the pH 2.5 dissociation condition (t-test; P<0.001). Interestingly, samples from mice inoculated with the DM Aβ peptide showed only a slight increase after dissociation which was not statistically significant. Importantly, under both conditions, the sera from mice inoculated with the DM peptide have substantially greater antibody titers than mice inoculated with WT Aβ or VLP AB.

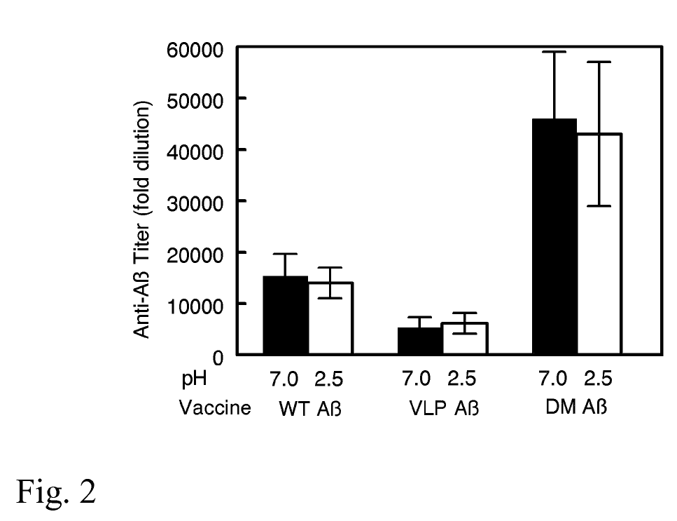

Surprisingly however, in sera from non-transgenic mice, which do not have human Aβ1-42 in their circulation, there is no effect of the acid dissociation treatment on the ELISA values (FIG. 2). This indicated the increase was due to dissociation of Aβ from the antibody in the low pH condition rather than some nonspecific modification of the antibodies caused by transient incubation at low pH. It also implies that recovery of the antibody activity is relatively intact after acid dissociation. Again, the titers in mice vaccinated with the DM peptide are significantly greater than the titers observed in mice administered the other two vaccines.

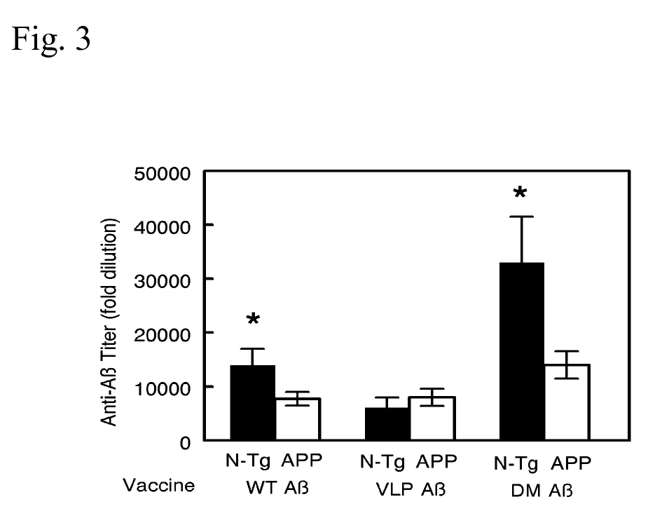

The anti-Aβ antibody titers in transgenic and non-transgenic mice were compared after acid dissociation (FIG. 3). Importantly, even after unmasking the antibodies with the acid dissociation, the antibody titers in transgenic mice were lower than the levels found in the non-transgenic animals for mice administered the WT or DM Aβ vaccines (t-test, P<0.05). However, for mice administered the VLP Aβ vaccine, the antibody titers were equivalent in the transgenic and non-transgenic mice. These data argue that some self-tolerance does exist in the APP transgenic mice above and beyond the antibody masking by circulating human Aβ, and that this self-tolerance can be overcome using the VLP Aβ conjugated vaccine. It is important to note that the amount of Aβ in the VLP vaccine is 5% of that used in the WT and DM vaccines. Higher VLP doses would be likely to cause considerably greater antibody titers in both genotypes. These results are consistent with other data using the VLP approach to overcome self-tolerance.

The data shown here indicate that at moderate antibody titers, circulating human Aβ in APP transgenic mice can interfere with the measurement of antibody titers in standard ELISA assays. At high anti-Aβ antibody titers, as found in the DM vaccinated mice, the antibody concentration appears to exceed the Aβ concentration sufficiently that the masking effects of Aβ are less significant in evaluating the titer. In support of this argument, the effects of acid dissociation on sera from mice vaccinated just twice with DM Aβ were examined when titers were roughly 1:800 without acid dissociation. Here, the dissociation increased the titers to roughly 1:6400, an 8 fold elevation. Thus, the failure of acid incubation to increase titers for the DM peptide vaccinated mice shown in FIG. 1 is likely due to the excess of antibody over circulating antigen, rather some alteration in the antibody or its epitope associated with this vaccine. When coating the ELISA plate with the DM Aβ instead of the Aβ1-42, similar titers are found implying the antibodies raised with the DM vaccine are not specific to that antigen. When titers are lower after just two inoculations, acid dissociation does successfully unmask antibody using this vaccine.

Three findings were found from these data. First, circulating Aβ can interfere with anti-Aβ antibody ELISA assays. The amount of circulating Aβ is known to vary considerably in AD patients. In at least some transgenic mouse models, anti-Aβ antibodies are known to increase the amounts of Aβ in the circulation. It will be important in clinical studies evaluating the anti-AB antibody content in circulation to insure that the methods used for detection are not confounded by Aβ peptide that might be bound to these proteins. Second, the DM peptide is considerably more antigenic than the WT Aβ peptide. The primary disease in patients carrying the Dutch APP mutation is an accumulation of vascular amyloid, with few parenchymal deposits. This is not unlike the pathology reported in the single autopsy case of a patient vaccinated with Aβ during a clinical trial. In two APP mouse models, anti-Aβ antibodies have been reported to increase the frequency of microhemorrhage. These results suggest that evaluation of patients with the Dutch mutation for the possibility of high anti-Aβ antibody titers is warranted. Finally, the VLP vaccine appears to evade the mechanisms restricting formation of antibodies to self antigens. As APP is a self-protein in AD patients, the use of this vaccine formulation may prove superior to more traditional immunization approaches.

FIGS. 1-3 illustrate antibody level detected from vaccinated APP transgenic mice and its littermate before and after treatment with dissociation buffer.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be the to fall therebetween. Now that the invention has been described,

What is claimed is:

1. A method of dissociating an antibody from a corresponding antigen in an antibody/antigen complex comprising the steps of:
obtaining a sample from a subject containing an antibody/antigen complex;
diluting the sample with a dissociation buffer;
incubating the sample; and
centrifuging the sample.

2. The method of claim 1 wherein the antibody/antigen complex comprises the amyloid-beta (Aβ)/anti-amyloid beta (anti-Aβ) complex.

3. The method of claim 1 wherein the sample is diluted with the dissociation buffer to a ratio of about 1:1000.

4. The method of claim 1 wherein the sample is diluted with the dissociation buffer to a final volume of about 500 μl

5. The method of claim 1 wherein the dissociation buffer is PBS buffer.

6. The method of claim 5 wherein the PBS buffer contains about 1.5% BSA and 0.2 M glycine-acetate pH 2.5)

7. The method of claim 1 wherein the sample is incubated for about 20 minutes at approximately room temperature.

8. The method of claim 1 wherein the sample is centrifuged twice.

9. The method of claim 8 wherein the sample is centrifuged first time at about 8,000 g.

10. The method of claim 9 wherein the sample is centrifuged for about 20 minutes at approximately room temperature.

11. The method of claim 9 wherein the sample is centrifuge a second time at about 1,000 g.

12. The method of claim 11 wherein the sample is centrifuged for about 3 minutes.

13. An assay for determining the presence of anti-Aβ antibodies in a sample, comprising the steps of:
dissociating the Aβ antigen from the anti-Aβ antibody present in the sample;
adjusting the pH of the sample to about 7.0;
adjusting the volume of the sample;
determining the limiting antibody concentration.

14. The method of claim 13 wherein the Aβ antigen is separated from the anti-Aβ antibody using the method of claim 1.

15. The method of claim 13 wherein the pH of the sample is adjusted using about 15 μl of about 1 M Tris Buffer (pH about 9.0).

16. The method of claim 13 wherein the volume of the sample is adjusted to an amount approximately equal to the volume of the original sample and dissociation buffer.

17. The method of claim 16 wherein the volume of the sample is adjusted to about 500 μl.

18. The method of claim 16 wherein the volume of the sample is adjusted with ELISA dilution buffer.

19. The method of claim 18 wherein the ELISA dilution buffer substantially comprises PBS with about 1.5% BSA and about 0.1% Tween-20, pH about 7.0.

20. The method of claim 13 wherein the limiting antibody concentration is determined using ELISA analysis.

21. A method of dissociating an antibody from a corresponding antigen in an amyloid-beta (Aβ)/anti-amyloid beta (anti-Aβ) complex comprising the steps of:
obtaining a sample from a subject containing an antibody/antigen complex;
diluting the sample with a PBS dissociation buffer, wherein the PBS Buffer contains about 1.5% BSA and about 0.2 M glycine-acetate pH 2.5, to a final volume of about 500 μl wherein the ratio of dilution is about 1:1000;
incubating the sample for about 20 minutes at approximately room temperature; and
centrifuging the sample a first time at about 8,000 g for about 20 minutes at approximately room temperature; and
centrifuging the sample a second time at about 1,000 g for about 3 minutes;

22. An assay for determining the presence of anti-Aβ antibodies in a sample, comprising the steps of:
dissociating the Aβ antigen from the anti-Aβ antibody present in the sample using the method as substantially stated in claim 1;
adjusting the pH of the sample to about 7.0 using about 15 μl of about 1 M Tris Buffer (pH about 9.0);
adjusting the volume of the sample with ELISA dilution buffer, comprising PBS with about 1.5% BSA and about 0.1% Tween-20 with a pH of about 7.0, to about 500 μl, an amount approximately equal to the volume of the original sample and dissociation buffer; and
determining the limiting antibody concentration in the sample using ELISA analysis.
